(19) United States
(12) Patent Application Publication
(10) Pub. No.: US 2005/0192272 A1
(43) Pub. Date: Sep. 1, 2005

(54) Substituted benzodiazepines as inhibitors of the chemokine receptor CXCR4

(76) Inventor: Garland Marshall, Clayton, MO (US)

Correspondence Address: James B. Surber, Esq.;Suite 3600, Bryan Cave,LLP, 211 N. Broadway, St. Louis, MO, 63102, US

(21) Appl. No.: 11/021,345

(22) Filed: Dec. 22, 2004

Related U.S. Application Data
(60) Provisional application No. 60/531,725, filed on Dec. 22, 2003.

(51) Int. Cl. A61K 31/5513

(52) U.S. Cl. 514/221

(57) ABSTRACT

Compositions and methods that can be used to prevent HIV infection are described. In particular, methods for inhibiting HIV infectivity are provided, which comprise administering to a patient a therapeutically-effective amount of at least one CXCR4 receptor-binding composition described herein. The CXCR4 receptor-binding compositions useful in the present invention include certain benzodiazepines, naphthylbenzodiazepines and analogs thereof.

### CROSS-REFERENCE TO RELATED APPLICATIONS

This invention claims priority under 35 U.S.C. §119(e) from U.S. Provisional Application Ser. No. 60/531,725, filed Dec. 22, 2003, entitled, “Substituted Benzodiazepines as Inhibitors of the Chemokine Receptor CXCR4.” The entire disclosure of U.S. Provisional Application Ser. No. 60/531,725 is incorporated herein by reference.

### FIELD OF THE INVENTION

The present invention relates to the field of therapeutics. More specifically, the invention relates to methods and compositions that can be used to inhibit HIV infectivity and treat Acquired Immunodeficiency Syndrome (AIDS), as well as other diseases such as cancers, asthma, arthritis, etc. in which the CXCR4 receptor and its chemokine ligand CXCL12 are implicated. In particular, the invention relates to certain benzodiazepines and naphthylbenzodiazepines, analogs thereof and pharmaceutically-acceptable salts thereof as inhibitors of the chemokine receptor CXCR4, which can be used to inhibit HIV infectivity and treat AIDS, as well as other diseases such as cancers, asthma, arthritis, etc. in which the CXCR4 receptor and its chemokine ligand CXCL12 are implicated.

### BACKGROUND OF THE INVENTION

The medications that are currently available to limit Human Immunodeficiency Virus (HIV) infectivity and treat Acquired Immunodeficiency Syndrome (AIDS) target specific proteins that are encoded by the viral genome, such as reverse transcriptase, integrase, gp120, gp41, or HIV protease. During the early years of research into therapeutics that may be used to fight HIV, this was a seemingly logical strategy as such proteins are uniquely expressed in patients during HIV infection. Unfortunately, however, the significant mutation and multiplication rate of HIV has resulted in the rapid development of resistance to such therapeutics during treatment. In fact, the limited period of effectiveness of such medications has led to the development of a variety of different inhibitors for many of the same viral proteins, and, of course, the use of polypharmacy, e.g., cocktails of reverse transcriptase/protease inhibitors, in an attempt to minimize the rate of resistance development.

In light of the foregoing, it has been suggested that the development of resistance to HIV therapeutics may be curtailed by, preferably, providing patients with medications that target conserved human proteins, which are essential for viral replication, rather than rapidly-mutating viral proteins. This strategy is particularly attractive given the extensive body of information that has been generated from studies of the mechanisms by which HIV enters a target cell. In particular, the cell-surface proteins/receptors involved in HIV entry have been identified, namely, the CD4 protein and the G-protein coupled receptors (GPCR) CCR5 and CXCR4.^{[1][2][3][4]}

In addition, it has been shown that strains of HIV have mutated to interact preferentially with CCR5 co-receptors for infection of macrophages, i.e., R5-strains, and CXCR4 co-receptors for infection of T-cells, i.e., X4-strains. In fact, recent studies suggest that initial infection of sexually transmitted HIV occurs, almost exclusively, through the R5-strains of HIV, which target the CCR5 chemokine receptor on the cell surface of macrophages. Mutation of certain viral proteins of the R5-strain results in the conversion to the X4-strains, which, recent evidence suggests,^{[5]} is highly correlated with HIV-positive patients succumbing to the Acquired Immunodeficiency Syndrome (AIDS) and the massive T-cell apoptosis that occurs in connection therewith. Accordingly, there is a substantial and continuous need for novel therapeutics that target conserved human proteins that are essential to HIV entry and, in particular, block X4-strain HIV infectivity to discourage the onset of AIDS in patients infected with HIV.

The chemokine receptor CXCR4 is a seven-transmembrane, GPCR of the Cys-X-Cys subclass of the rhodopsin family of GPCRs. Among its several physiological roles, CXCR4, when expressed on the surface of the cell, interacts with its natural ligand, CXCL12 (stromal-cell derived factor, SDF-1), to elevate intracellular calcium levels and is involved with chemotaxis of T-cells of the immune system. In addition to its normal role as the chemokine receptor for CXCL12, CXCR4 serves as a binding and entry point for HIV into cells that co-express the protein CD4 on their surface.

The chemokine CXCL12 is the only physiological agonist of the CXCR4 receptor that has been identified to date. CXCL12 is expressed and constitutively secreted by almost all cell types and is important in the migration and development of hematopoietic cells. The CXCR4 receptor and CXCL12 have also been associated with pathogenesis of rheumatoid arthritis and in a variety of cancers. In particular, angiogenesis and metastasis of several different cancer types have been shown to be associated with elevated CXCR4 expression.^{[6-8]}

Several compounds have been shown to block HIV X4-strain infectivity as antagonists of the CXCR4 receptor.^{[9]} Many of these CXCR4 inhibitors have been discovered by randomly screening compound libraries for inhibition of CXCL12 binding,^{[10]} inhibition of HIV X4-strain infectivity and/or inhibition of calcium influx in cells expressing the CXCR4 receptor when challenged with CXCL12. More interestingly, several of these inhibitors were discovered by functional antiviral assays before the existence of the CXCR4 receptor and its physiological role, or its significance in HIV infectivity, were known.^{[11]}

Non-limiting examples of CXCR4 inhibitors known in the art include a variety of highly basic peptides, such as N-terminal segments of CXCL12 (SDF-1) itself, ALX40-4C and the natural product polyphemusin, as well as a series of bicyclams^{[12][13][14]} and monocyclams^{[15]} as represented by AMD3100.^{[16-18]} The structures of such inhibitors are shown below:

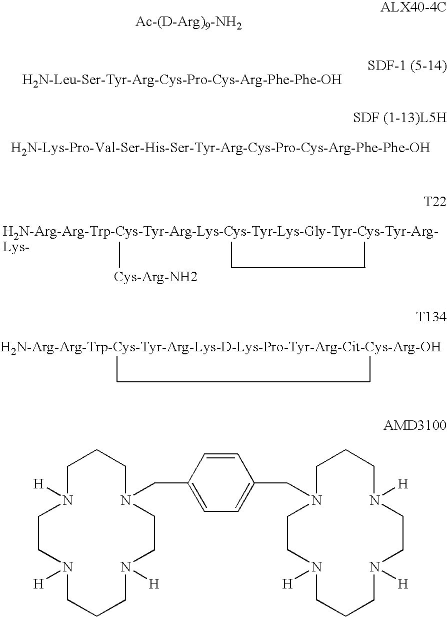

During Phase I/II clinical trials conducted by AnorMED, Inc. (Langley, British Columbia, Canada), the AMD3100 inhibitor exhibited certain cardiac toxicity properties and limited effectiveness, which resulted in the termination of that trial. The toxicity, however, was not necessarily the result of AMD3100 binding to CXCR4 receptors in the heart. Rather, the toxicity most likely can be attributed to unidentified side-interactions of AMD3100 with other physiologically-important systems for cardiac function, i.e., a compound class-based toxicity.

This hypothesis is supported by the month-long clinical study of ALX40-4C, conducted in 1995 prior to the discovery of HIV co-receptors, which did not exhibit any acute cardiac toxicity. In particular, ALX40-4C completed a one-month Phase I/II clinical study with no adverse side effects. Thus, as stated, the acute toxicity observed in connection with AMD3100 is most likely a property of that class of molecules and should not be associated with other CXCR4 antagonists.

Another compound that targets CXCR4 receptors is KRH-1636 (structure shown below), which is absorbed from the duodenum in rats. KRH-1636 (IC₅₀=13 nM) was optimized from a lead compound that was discovered by screening over 1000 compounds from Kureha Chemical Industry (Chuo-ku, Tokyo). KRH-1636 is, in essence, a modification of the dipeptide Acyl-Arg-Gly(Naphthalene)-OH having two chiral centers with the acyl group being a para-substituted benzoic acid, and the Gly(Naphthalene) carboxy being replaced by a methyl group.

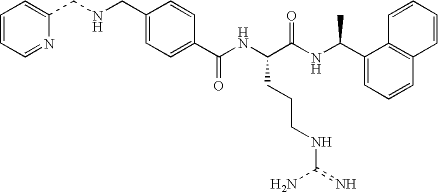

Still further, a cyclic pentapeptide FC131 c(Nal-Gly-tyr-Arg-Arg) (Nal=naphthylalanine, tyr=D-Tyr, following an accepted convention) has been described, which has an IC₅₀ of 4 nM in inhibiting the binding of CXCL12 to CXCR4. This molecule was discovered by screening a library of cyclic pentapeptides. The library was designed based on SAR (Structure-Activity Relationship) data from studies involving the highly selective CXCR4 antagonist T-22 ([Tyr^{5,12}, Lys⁷]-polyphemusin II) and its shorter analogs, T134, T140 and TC14012, which strongly inhibit multiplication of X4-strains of HIV through specific binding to the CXCR4 receptor. In fact, T140 and TC14012 exhibited the highest level of anti-HIV activity and antagonism of all the CXCR4 antagonists reported to date, which has since been matched by FC131.

While others have employed random screening methods to develop novel CXCR4 inhibitors, including fragments of the natural ligand CXCL12, the inventor has found that structure-based drug design is a far superior method of identifying novel CXCR4 inhibitors. Specifically, the inventor has discovered that based, in part, on information related to the three-dimensional orientation of certain pharmacophoric groups of FC131 and KRH-1636 when bound to the CXCR4 receptor, additional inhibitors can be developed. In particular, the inventor has found that additional CXCR4 inhibitors can be designed by employing certain scaffolds that, preferably, are capable of correctly orienting certain pharmacophoric groups, such as two guanidinium groups or surrogates, and one or more aromatic rings, to the active site of CXCR4.

The inventor has shown, for example, that benzodiazepines, a class whose synthetic chemistry is well-established, some of which may be synthesized from amino acid synthons, can be used as scaffolds for the three-dimensional orientation of such pharmacophoric groups to the active site of CXCR4. Thus, the present invention provides novel compositions (and methods of using the same) that target the conserved human CXCR4 receptor, which is essential to HIV X4-strain entry, and, more importantly, may serve as effective therapeutics to discourage the onset of AIDS or treat diseases associated with elevated levels of expression of either CXCL12 or CXCR4.

### SUMMARY OF THE INVENTION

The present invention provides compositions that are capable of binding the CXCR4 receptor on mammalian cells, which can be used to inhibit or reduce HIV infectivity. In addition, such compositions can be used to prevent the interaction of CXCL4 with CXCR4 in disease states (cancer, asthma, arthritis, inflammation, etc.) where expression of either CXCL4 or CXCR4 is elevated and/or activation of the CXCR4 system contributes to the disease.

In one embodiment, certain benzodiazepine rings are provided, which are modified to exhibit at least two positively charged guanido, benzamidino or their surrogate groups. The guanido groups, or their surrogates, are, preferably, positioned in a way that facilitates interaction with two or more carboxyl groups of Asp171, Asp262 and/or Glu288 of the CXCR4 receptor. In addition, the benzodiazepine rings of this preferred embodiment are further modified to exhibit at least one substituent comprising a planar aromatic or hydrocarbon ring and their analogs.

In a second embodiment, certain naphthylbenzodiazepines are provided as inhibitors of the CXCR4 receptor. In particular, naphthylbenzodiazepines are provided with at least two guanido, or their surrogates, substituents on the benzodiazepine ring. Such compositions represent an additional class of tripodal scaffolds with appropriately oriented substituents, which can effectively bind the CXCR4 receptor and, preferably, inhibit or reduce HIV infectivity, and treat other diseases associated with CXCL12 or CXCR4.

In related embodiments, certain pharmacologic formulations are provided, which can be used to inhibit or reduce HIV infectivity. Such pharmacologic formulations may comprise one or more of the CXCR4-binding compositions described herein (or their therapeutically-active non-toxic salt forms), one or more acceptable carriers and/or other anti-viral agents (which are currently available or discovered hereafter).

In further embodiments of the present invention, methods of using the pharmacologic compositions described herein to block the CXCR4 receptor are provided, which, preferably, can be employed to inhibit or reduce HIV infectivity in a patient, and treat other diseases associated with CXCL12 or CXCR4 pathogenesis. The methods of the present invention comprise administering to a patient an effective amount of at least one pharmacologic composition described herein.

### DESCRIPTION OF THE FIGURES

FIG. 1: Graphical representation of KRH-1636 conformers that can overlap three of its pharmacophoric groups with those of FC131. Arginine/surrogate groups are oriented top and left, respectively; naphythyl group is oriented to the bottom right. Two methods (systematic search and a genetic algorithm) were used to generate the model of the KRH-1636 conformers shown.

FIG. 2: (A) Graphical representation of one of many KRH-1636 and FC131 overlaps, which was generated using a genetic algorithm. (B) Graphical representation of one of many KRH-1636 and FC131 overlaps, which was generated using a systematic search. In both A and B, the Arginine/surrogate overlap is shown near the top; the Arginine/Arginine overlap is shown in the middle; and the naphthyl/naphthyl overlap is shown near the bottom of the graphics.

FIG. 3: Model of AMD3100 (stick figure) in binding pocket of CXCR4 (surface) with three side-chain carboxyl groups (Asp171, Asp262 and Glu288) responsible for affinity (indicated by red oxygen atoms).

FIG. 4: Model of the bis-guanido-benzodiazepine, GM2812 (stick figure), in binding pocket of CXCR4 (surface) with three side-chain carboxyl groups (Asp171, Asp262 and Glu288) responsible for affinity for AMD3100 (indicated by red oxygen atoms) interacting with the two guanido groups.

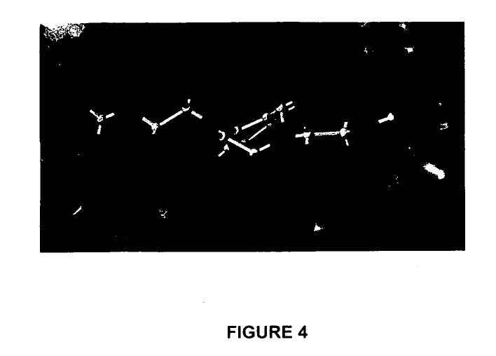

FIG. 5: Model of GM2813 (stick figure) in binding pocket of CXCR4 (surface) with three side-chain carboxyl groups (Asp171, Asp262 and Glu288) responsible for affinity for AMD3100 (indicated by red oxygen atoms) interacting with the two guanido groups. The naphthyl ring of GM2813 is further shown to interact with the hydrophobic pocket in this site of the CXCR4 receptor.

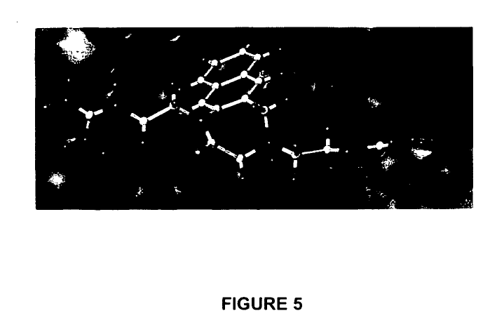

### DETAILED DESCRIPTION OF THE INVENTION

The following will describe in detail several preferred embodiments of the present invention. These embodiments are provided by way of explanation only, and thus, should not unduly restrict the scope of the invention. In fact, those of ordinary skill in the art will appreciate upon reading the present specification and viewing the present drawings that many variations and modifications of the invention may be employed, used and made without departing from the scope and spirit of the invention.

The contents of each of the references discussed in this specification, including the references cited, are herein incorporated by reference in their entirety. In particular, each U.S. patent and U.S. patent application publication discussed and/or cited in this specification are herein incorporated by reference in their entirety.

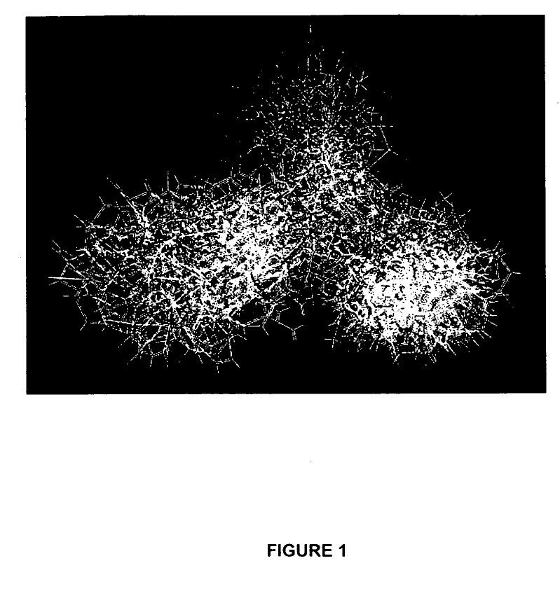

The compositions of the present invention were discovered through a process of rational drug design. In particular, the compositions of the present invention were designed based on the premise that both KRH-1636 and FC131 bind to the same site of the CXCR4 receptor through the functional groups that the two compounds have in common—oriented in a similar manner. The geometric feasibility of this premise was verified by molecular modeling using the SYBYL suite of programs (Tripos, Inc., St. Louis, Mo.). To that end, a set of conformers (shown in FIG. 1) of KRH-1636 were identified, wherein the guanidinium group of KRH-1636 could overlap one of the guanidinium groups of FC131; the 2-methylaminopyridine portion of KRH-1636 could overlap the guanidinium surrogate group of FC131; and the naphthyl groups of the two compounds could simultaneously overlap. As shown in FIG. 1, many conformers were identified, which should be expected for such flexible compounds.

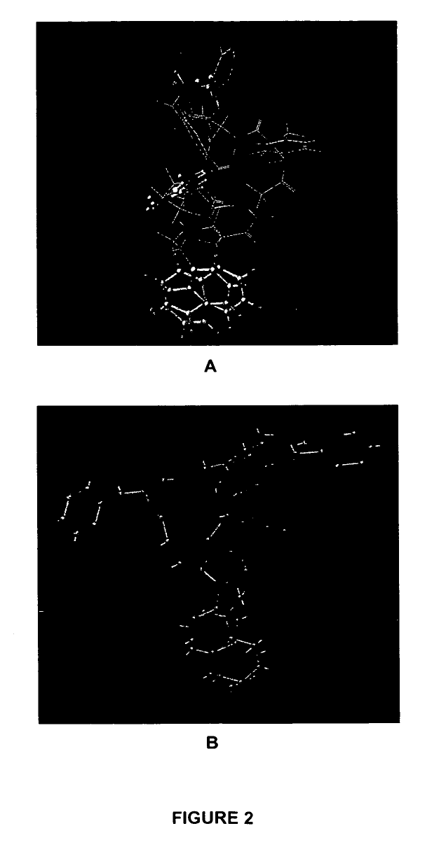

The foregoing set of conformers produced a low-resolution model of receptor-bound conformations of KRH-1636. This model provided a suitable three-dimensional template for designing benzodiazepines with a planar aromatic substituent and two guanidinium (or guanido surrogate) substituents correctly oriented in space to interact with complementary sites on the CXCR4 receptor. Non-limiting examples of two different three-dimensional overlaps of the three functional groups of KRH-1636 and FC131 are shown in FIG. 2, which were generated using two different approaches of searching conformational space (systematic search and genetic algorithm).

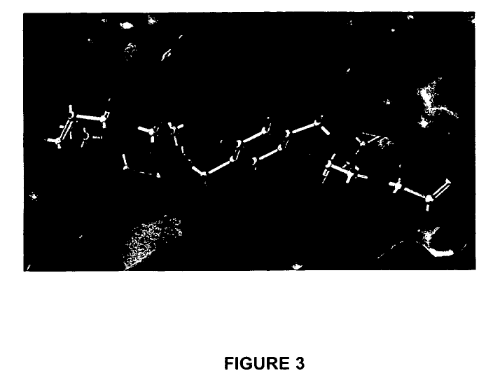

After generating the model of receptor-bound conformations of KRH-1636, a three-dimensional map of the binding site of CXCR4 was prepared by threading the CXCR4 sequence based on homology through the crystal structure of dark-adapted rhodopsin and optimizing the packing of amino acid side chains. Several papers have described the amino acid residues of the CXCR4 receptor that are most critical for binding the AMD3100 antagonist. In particular, Asp262, Asp271 and Glu288 were identified by mutational analysis as essential binding points. Thus, the Asp262, Asp271 and Glu288 residues were targeted as binding sites for the two positively charged cyclam ends of AMD3100 to generate the model of the bound complex of AMD3100 and CXCR4 shown in FIG. 3. Additional modeling has shown that KRH-1636 can bind to the same site and insert its naphythyl group into a deep hydrophobic pocket, which is located adjacent to the three-dimensional space occupied by Asp262, Asp271 and Glu288.

The present invention provides compositions that are capable of binding the CXCR4 receptor in a similar manner at the same site. More specifically, based on the modeling studies described above, the compositions described herein were designed to fit within the binding site of the CXCR4 receptor, i.e., to be volume-compatible, and, moreover, be capable of orienting certain pharmacophoric groups in the relative proximity of Asp262, Asp271 and Glu288.

In one preferred embodiment of the present invention, certain benzodiazepines are provided that are capable of binding the CXCR4 receptor, which can be used to inhibit HIV infectivity, or treat other CXCR4/CXCL12 associated diseases. The compositions of this embodiment represent a class of tripodal scaffolds with appropriately oriented substituents that can bind to the CXCR4 receptor. Specifically, the compositions of this embodiment comprise a benzodiazepine ring, with three substitutents that are correctly oriented to bind and/or interact with the appropriate residues of the CXCR4 receptor, namely, Asp262, Asp271 and Glu288. Non-limiting examples of these substitutents include planar aromatic/hydrocarbon rings (benzyl, naphthyl, cyclohexyl, cyclopentyl, etc.) with appropriate substitutents, as well as substitutents bearing guanido groups (or their surrogates and/or “prodrug” modifications to enhance bioavailability).

In this embodiment, a benzodiazepine ring is modified to exhibit, preferably, at least two positively charged guanido groups. The guanido groups are positioned to interact with one or more of the carboxyl groups of Asp171, Asp262 and/or Glu288 of the CXCR4 receptor. Still further, the diazepine ring is modified to exhibit at least one substituent comprising a planar aromatic ring such as a benzodiazepine. The aromatic ring is, preferably, capable of orienting into the hydrophobic pocket of the CXCR4 receptor, which is located adjacent to the same three-dimensional space occupied by the carboxyls of Asp171, Asp262 and Glu288 of the CXCR4 receptor.

GM2812 (N-{2-[4-(2-Guanidino-ethyl)-2-exo-2,3,4,5-tetrahydro-benzo[e][1,4,]diazepin-1-yl]-ethyl}-guanidine) represents a non-limiting example of this preferred embodiment. As shown below, GM2812 exhibits two guanido groups on arginine-like substituents and an aromatic ring, which are correctly oriented to bind and/or interact with the appropriate residues of the CXCR4 receptor.

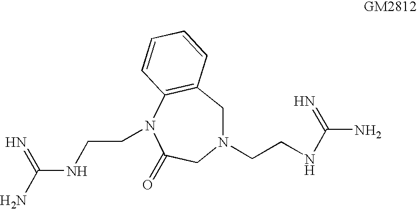

GM2812 can be modified in various ways known the art to (i) increase its ability to bind the CXCR4 receptor and/or (ii) impart certain “prodrug” attributes, e.g., to mitigate toxicity, increase bioavailability, etc. A more detailed description of such “prodrug” attributes may be found in U.S. Pat. Nos. 6,596,729 and 6,506,770, the disclosures of which are incorporated herein by reference. Such derivatives of GM2812 should be understood by those skilled in the art to fall within the scope of the present invention. Non-limiting examples of the various sites at which GM2812 can be modified are shown below.

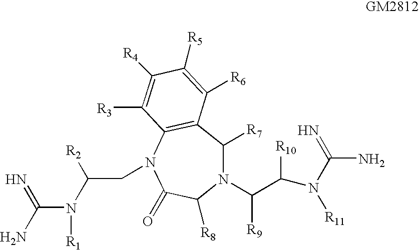

As used herein, all R-groups shown or referenced indicate that the corresponding CH₃, —(CH₂)ₙ—, CH or NH moiety can, optionally, be modified as described. That is, each of the CXCR4-binding compositions of the present invention can, optionally, be unsubstituted or substituted at, for example, one or more of the locations labeled Rₙ. With respect to GM2812, for example, R₁, R₂ and R₇₋₁₁ each can be a hydrocarbon, saturated or unsaturated, NH or NH₂ group. In addition, R₁, R₂ and R₇₋₁₁ each can be a mono- or di(C₁₋₆alk-)amino group—depending, of course, on the valence of the modified atom. As used herein, “C₁₋₆alk-” shall mean straight or branched chain, saturated or unsaturated, hydrocarbon groups having 1 to 6 carbon atoms. Still further, for example, R₃₆ can be a hydrocarbon, saturated or unsaturated (potentially aromatic), which is generally hydrophobic, up to about C₁₂.

All of the CXCR4-binding compositions described and/or shown herein to be useful in the present invention may also exist in their tautomeric forms. Of course, the tautomeric forms of such structures are intended to be included within the scope of the present invention. The present invention should further be understood by those skilled in the art to encompass pharmaceutically-acceptable salts of the CXCR4-binding compositions described herein, as well as all stereochemically isomeric forms thereof, which include all diastereomers and enantiomers of said compositions.

Additional preferred embodiments of the present invention include GM2822 (N-{2-[3-(2-Guanidino-ethyl)-2,5-dioxo-2,3,4,5-tetrahydro-benzo[e][1,4]diazepin-1-yl]-ethyl}-guanidine), GM2824 (4-[3-(2-Guanidino-ethyl)-2,5-dioxo-2,3,4,5-tetrahydro-benzo[e][1,4]diazepin-1-ylmethyl]-benzamidine) and GM2825 (N-(4-Carbamimidoyl-benzyl)-4-guanidino-N-methyl-2-phenylamino-butyramide). The structures of GM2822, GM2824 and GM2825 are shown below.

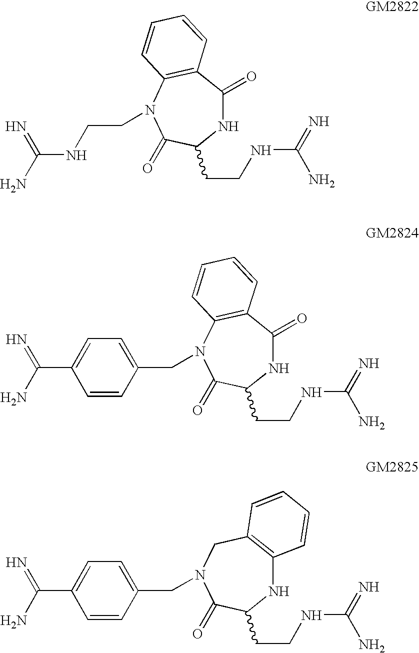

Similarly, GM2822, GM2824 and GM2825 can be modified in various ways known the art to affect CXCR4-binding activity and/or impart certain “prodrug” attributes. For purposes of illustration only, examples of the various sites at which GM2822, GM2824 and GM2825 can be modified are shown below.

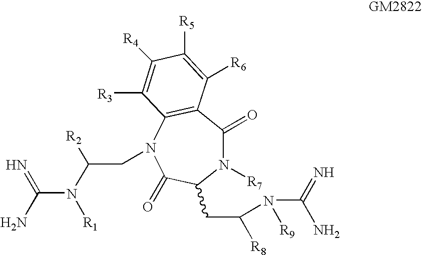

In this example, generally, R₁, R₂ and R₇₋₉ of GM2822 each can be a hydrocarbon, saturated or unsaturated, NH or NH₂ group. In addition, R₁, R₂ and R₇₋₉ each can be a mono- or di(C₁₋₆alk-)amino group. Still further, for example, R₃₋₆ can be a hydrocarbon, saturated or unsaturated (potentially aromatic), which is generally hydrophobic, up to about C₁₂.

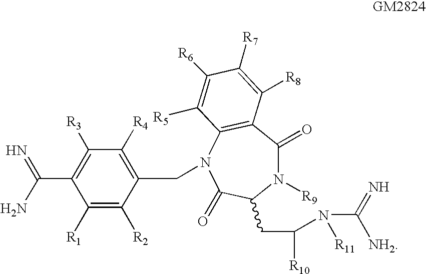

R₁₋₃ and R₉₋₁₁ of GM2824 each can be a hydrocarbon, saturated or unsaturated, up to about C₆, NH or NH₂ group. In addition, R₁₋₃ and R₉₋₁₁ each can be a mono- or di(C₁₋₆alk-)amino group. Still further, for example, R₅₋₈ can be a hydrocarbon, saturated or unsaturated (potentially aromatic), which is generally hydrophobic, up to about C₁₂.

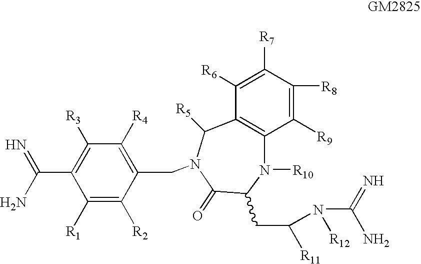

Similarly, R₁₋₃ and R₉₋₁₂ of GM2825 each can be a hydrocarbon, saturated or unsaturated, up to about C₆, NH, NH₂ or mono- or di(C₁₋₆alk-)amino group. Still further, for example, R₅₋₉ can be a hydrocarbon, saturated or unsaturated (potentially aromatic), which is generally hydrophobic, up to about C₁₂.

In a related, preferred embodiment of the present invention, certain naphthylbenzodiazepines are provided as inhibitors of the CXCR4 receptor. In particular, naphthylbenzodiazepines are provided with at least two guanido substituents on the benzodiazepine ring. Such molecules represent yet another class of tripodal scaffolds with appropriately oriented substituents that can bind to the CXCR4 receptor.

GM28 13 (N-{2-[4-(2-Guanidino-ethyl)-2-exo-2,3,4,5-tetrahydro-naphtho[2,3-e][1,4]diazepin-1-yl]-ethyl}-guanidine) represents a non-limiting example of this embodiment. As shown below, GM2813 comprises a naphthylbenzodiazepine ring, wherein the ring is modified to exhibit two guanido groups on arginine-like substituents.

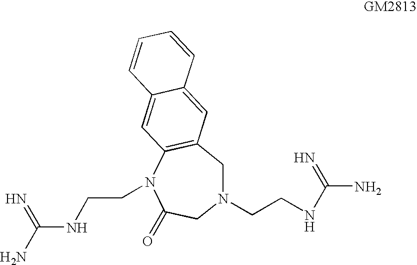

Still further, GM2823 (N-{2-[3-(2-Guanidino-ethyl)-2,5-dioxo-2,3,4,5-tetrahydro-naphtho[2,3-e][1,4]diazepin-1-yl]ethyl}-guanidine) represents another non-limiting example of this embodiment. As shown below, GM2823 also comprises a naphthylbenzodiazepine ring, wherein the ring is modified to exhibit two guanido groups.

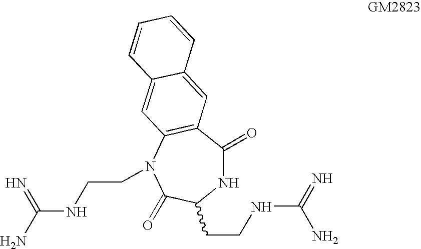

Similar to the benzodiazepine compositions of the present invention, the naphthylbenzodiazepines described herein can be modified to affect CXCR4-binding activity and/or to impart certain “prodrug” characteristics. Non-limiting examples of locations at which GM2813 and GM2823 can, optionally, be modified are shown below.

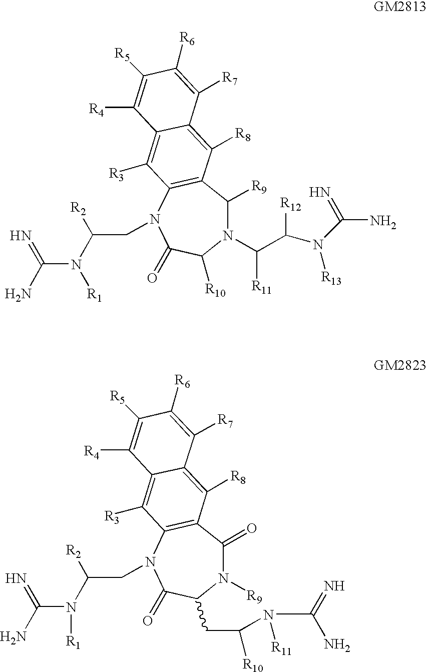

R₁, R₂ and R₁₁₋₁₃ of GM2813 each can be a hydrocarbon, saturated or unsaturated, up to about C₆, NH or NH₂ group. In addition, R₁, R₂ and R₁₁₋₁₃ each can be a mono- or di(C₁₋₆alk-)amino group. Still further, for example, R₃₋₈ can be a hydrocarbon, saturated or unsaturated (potentially aromatic), which is generally hydrophobic, up to about C₆. Similarly, R₁, R₂ and R₉₋₁₁ of GM2823 each can be a hydrocarbon, saturated or unsaturated, up to about C₆, NH, NH₂ or mono- or di(C₁₋₆alk-) amino group. Consistent with the foregoing, R₃₋₈ can be a hydrocarbon, saturated or unsaturated (potentially aromatic), which is generally hydrophobic, up to about C₆.

The CXCR4-binding compositions of the present invention can be synthesized using methods well-known in the art. Horton et al., for example, teaches certain methods that may, optionally, be employed to synthesize several compositions of the present invention.^{[19]} In particular, Horton et al. provides methods of generating the 1,4-benzodiazepin-2,5-diones of the present invention, namely, GM2822 and GM2824. As will be appreciated by those skilled in the art, GM2823 can also be generated using these procedures with slight modifications.

Specifically, Horton et al. teaches at least two strategies for the synthesis of GM2822, GM2824 and GM2823. The first method is based on the use of amino acid derivatives, whereas the second method comprises a four component Ugi reaction. The general method involving the use of amino acid derivatives, for example, is illustrated below:

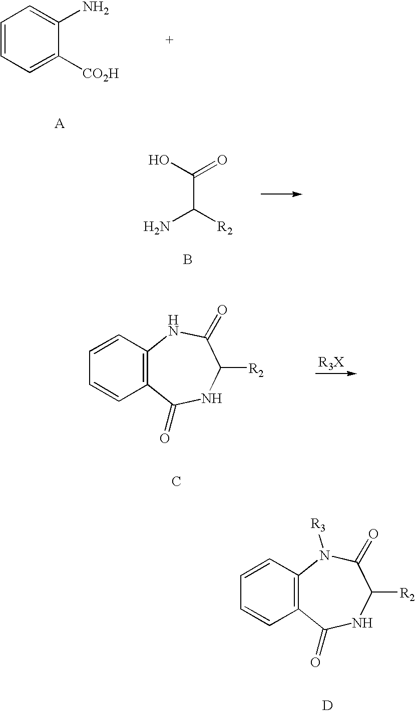

In this method, anthranilic acid (or an anthranilic acid derivative) (A), which is often N-protected or masked as a nitro or azido group, and an amino acid (or amino acid derivative) (B) are condensed to produce the 1,4-benzodiazepin-2,5-dione ring (C). As those skilled in the art will appreciate, a plethora of different side chains can, optionally, be used at R₂ in (B) to generate the desired substituent at the corresponding location in the end product (D). The 1,4-benzodiazepin-2,5-dione ring can then be treated with an alkylating agent (R₃X) to produce (D). Horton et al. provides that, in most cases, a C-terminal protected amino acid can be used. The alkylating step is, generally, amenable to many different alkylating agents, which should be selected, of course, in accordance with the desired substituent at the corresponding location in the end product (D).

GM2822 can be synthesized using this method, for example, by condensing anthranilic acid (A) with an arginine-like derivative (B₁) to produce the 1,4-benzodiazepin-2,5-dione ring (C₁). As stated, this ring (C₁) can be treated with an alkylating agent (R₃X), which, preferably, comprises a protected arginine surrogate, to attach the desired guanido substituent at R₃. This method is illustrated below:

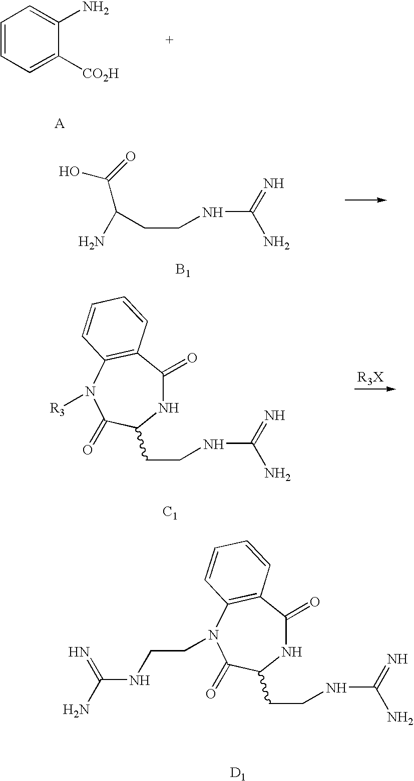

The method outlined above can also be used, for example, to synthesize GM2824. In this case, of course, a different alkylating agent (R₃X) is used. In general, any alkylating agent (R₃X) can be used, which produces the desired 4-ethyl-benzamidine substitution at R₃.

In addition, several methods currently exist for synthesizing the benzodiazepines and naphthylbenzodiazepines of the present invention, which are substituted on the two nitrogen atoms. In fact, U.S. patent application publication No. 2003/0186969, the disclosure of which is incorporated herein by reference, provides numerous routes toward synthesizing various benzodiazepines, which are substituted on the two nitrogen atoms. Those of ordinary skill in the art will appreciate that several of the methods described therein can be modified to produce, for example, GM2812 and 2813.

Still further, the naphthylbenzodiazepines of the present invention can be synthesized using the general methods described above, with only slight modifications. In particular, the methods are modified to employ naphthyl starting material rather than benzyl. GM2823, for example, can be generated using a similar method as that outlined above for GM2822, wherein (A) comprises a naphthyl starting material (an anthranilic acid derivative), such as 3-amino-naphthalene-2-carboxylic acid:

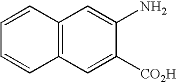

The CXCR4-binding compositions of the present invention may, optionally, be converted to their therapeutically-active non-toxic acid salt forms by treatment with appropriate acids. Such acids include inorganic acids, e.g., hydrochloric, hydrobromic and the like acids, sulfuric acid, nitric acid, phosphoric acid and the like; or organic acids, such as acetic, propanoic, hydroxyacetic, 2-hydroxypropanoic, 2-oxo-propanoic, ethanedioic, propanedioic and the like acids. Of course, the salt forms can be converted into the free base form by treatment with alkali. The pharmaceutically-acceptable acid salts of the present invention also comprise the solvates that the CXCR4-binding compositions of the present invention may form, which, of course, are included within the scope of the present invention. Non-limiting examples of such solvates are hydrates, alcoholates and the like.

The pharmacologic compositions of the present invention may be formulated in various ways known in the art for administration purposes. To prepare the pharmaceutical compositions of the present invention, an effective amount of the particular compound, in base or acid salt form, as the active ingredient is combined with one or more pharmaceutically-acceptable carriers. Numerous carriers exist that are readily accessible and well-known in the art, which may be employed to generate the preparation desired.

The pharmacologic compositions described herein are, preferably, in unitary dosage form suitable, for example, for administration orally, percutaneously or by parenteral injection (including subcutaneous, intramuscular, intravenous and intradermal). In preparing the compositions for oral dosage, for example, any of the pharmaceutical media known in the art can be used, such as water, glycols, oils, alcohols and the like in the case of oral liquid preparations such as suspensions, syrups, elixirs and solutions. When solid carriers are desired, starches, sugars, kaolin, lubricants, binders, disintegrating agents and the like can be used to prepare, for example, powders, pills, capsules and tablets. For parenteral compositions, acceptable carriers will usually comprise, primarily, sterile water, which can be supplemented with various solutes to, for example, increase solubility. Injectable solutions, for example, may be prepared in which the carrier comprises saline solution, glucose solution or a mixture thereof, which may contain certain well-known anti-oxidants, buffers, bacteriostats and other solutes that render the formulation isotonic with the blood of the intended recipient. For percutaneous administration, the carrier may optionally comprise a penetration enhancing agent and/or a suitable wetting agent.

In addition, the pharmacologic compositions described herein can, optionally, be combined with one or more other antiviral agents, such as certain well-known reverse transcriptase inhibitors, e.g., ddC, AZT and ddl, or other compounds that are known to inhibit the activity of other HIV proteins, such as protease, integrase and RNAasH. Still further, the compositions can, optionally, be mixed with certain well-known biological response modifiers, including interferon (α, β or γ), interleukin-2 and granulocyte-macrophage colony stimulating factor (GM-CSF) and the like.

In additional preferred embodiments of the present invention, methods of using the pharmacologic compositions described herein are provided to block the CXCR4 receptor and, preferably, inhibit or reduce HIV infectivity in a patient, or treat other CXCR4/CXCL12 associated diseases. A more detailed description of other CXCR4/CXCL12 associated diseases may be found, for example, in U.S. patent application publication No. 2003/0018046, the disclosure of which is incorporated herein by reference. The methods of the present invention comprise the steps of administering an effective amount of at least one pharmacologic composition described herein, a pharmaceutically-acceptable salt or a stereoisomeric form thereof.

Of course, those skilled in the art in treating viral diseases, or treating other CXCR4/CXCL12 associated diseases, could easily determine the effective therapeutic amount of any of the pharmacologic compositions described herein. In general, however, those skilled in the art will appreciate that such amounts, generally, may range from 0.1 mg/kg to 200 mg/kg body weight, and, in particular, from 1 mg/kg to 50 mg/kg body weight. The exact range, however, will largely depend upon the specific composition used, the dosage, the form of administration and/or information related to the intended recipient. It may be appropriate, of course, to administer the required dose at two, three, four or more sub-doses at appropriate intervals throughout the day.

The many aspects and benefits of the invention are apparent from the detailed description, and thus, it is intended for the following claims to cover all such aspects and benefits of the invention, which fall within the scope and spirit of the invention. In addition, because numerous modifications and variations will readily occur to those skilled in the art, the claims should not be construed to limit the invention to the exact construction and operation illustrated and described herein. Accordingly, all suitable modifications and equivalents should be understood to fall within the scope of the invention as claimed herein.

### REFERENCES

1. Cooley, L. A. and S. R. Lewin, HIV-1 cell entry and advances in viral entry inhibitor therapy. J. Clin. Virol., 2003. 26(2): p. 121-32.

2. Heveker, N., Chemokine receptors as anti-retroviral targets. Curr Drug Targets, 2001. 2(1): p. 21-39.

3. De Clercq, E., New anti-HIV agents and targets. Med Res Rev, 2002. 22(6): p. 531-65.

4. Este, J. A., Virus Entry as a Target for Anti-HIV Intervention. Curr. Med. Chem., 2003. 10(17): p. 1617-32.

5. Jekle, A., O. T. Keppler, E. De Clercq, D. Schols, M. Weinstein and M. A. Goldsmith, In vivo evolution of human immunodeficiency virus type 1 toward increased pathogenicity through CXCR4-mediated killing of uninfected CD4 T cells. J. Virol., 2003. 77(10): p. 5846-54.

6. Payne, A. S. and L. A. Cornelius, The role of chemokines in melanoma tumor growth and metastasis. J Invest Dermatol, 2002. 118(6): p. 915-22.

7. Murphy, P. M., Chemokines and the molecular basis of cancer metastasis. N Engl J Med, 2001. 345(11): p. 833-5.

8. Moore, M. A., The role of chemoattraction in cancer metastases. Bioessays, 2001. 23(8): p. 674-6.

9. Fujii, N., H. Nakashima and H. Tamamura, The therapeutic potential of CXCR4 antagonists in the treatment of HIV. Expert Opin Investig Drugs, 2003. 12(2): p. 185-95.

10. Ichiyama, K., S. Yokoyama-Kumakura, Y. Tanaka, R. Tanaka, K. Hirose, K. Bannai, T. Edamatsu, M. Yanaka, Y. Niitani, N. Miyano-Kurosaki, H. Takaku, Y. Koyanagi and N. Yamamoto, A duodenally absorbable CXC chemokine receptor 4 antagonist, KRH-1636, exhibits a potent and selective anti-HIV-1 activity. Proc Natl Acad Sci USA, 2003. 100(7): p. 4185-90.

11. De Clercq, E., The bicyclam AMD3100 story. Nat Rev Drug Discov, 2003. 2(7): p. 581-7.

12. U.S. Pat. No. 5,021,409 (Murrer, B. T. and D. A. Schwartz, Antiviral Cyclic Polyamines).

13. U.S. Pat. No. 5,583,131 (Bridger, G. J., et al., Aromatic-Linked Polyamine Macrocyclic Compounds with Anti-HIV Activity).

14. U.S. Pat. No. 5,698,546 (Bridger, G. J., et al., Cyclic Polyamines).

15. U.S. Pat. No. 6,506,770 (Bridger, G. J., et al., Antiviral Compounds).

16. Bridger, G. J., R. T. Skerij, S. Padmanabhan, S. A. Martellucci, G. W. Henson, M. J. Abrams, H. C. Joao, M. Witvrouw, K. De Vreese, R. Pauwels and E. De Clercq, Synthesis and structure-activity relationships of phenylenebis(methylene)-linked bis-tetraazamacrocycles that inhibit human immunodeficiency virus replication. 2. Effect of heteroaromatic linkers on the activity of bicyclams. J Med Chem, 1996. 39(1): p.109-19.

17. Joao, H. C., K. De Vreese, R. Pauwels, E. De Clercq, G. W. Henson and G. J. Bridger, Quantitative structural activity relationship study of bis-tetraazacyclic compounds. A novel series of HIV-1 and HIV-2 inhibitors. J Med Chem, 1995. 38(19): p. 3865-73.

18. Bridger, G. J., R. T. Skerlj, D. Thornton, S. Padmanabhan, S. A. Martellucci, G. W. Henson, M. J. Abrams, N. Yamamoto, K. De Vreese, R. Pauwels and et al., Synthesis and structure-activity relationships of phenylenebis(methylene)-linked bis-tetraazamacrocycles that inhibit HIV replication. Effects of macrocyclic ring size and substituents on the aromatic linker. J Med Chem, 1995. 38(2): p. 366-78.

19 Horton, D. A., G. T. Bourne and M. L. Smythe, The Combinatorial Synthesis of Bicyclic Privileged Structures or Privileged Substructures. Chem. Rev., 2003. 103: p. 893-930.

1. A method of inhibiting HIV infectivity, which comprises administering to a patient a therapeutically-effective amount of a composition selected from the group consisting of GM2812, GM2822, GM2824, GM2825, GM2813 and GM2823.
